(19) United States
(12) Patent Application Publication
(10) Pub. No.: US 2009/0326581 A1
(43) Pub. Date: Dec. 31, 2009

(54) EXPANDABLE SPACING MEANS FOR INSERTION BETWEEN SPINOUS PROCESSES OF ADJACENT VERTEBRAE

(76) Inventors: Geoffrey Harrison Galley, London (GB); James Bernard Allibone, Herts (GB); Mohammed Hamza Hilali Noordeen, London (GB); Benjamin Anthony Taylor, Herts (GB); Stewart Kenneth Tucker, London (GB)

Correspondence Address: STITES & HARBISON PLLC, 401 COMMERCE STREET, SUITE 800, NASHVILLE, TN, 37219, US

(21) Appl. No.: 12/294,152

(22) PCT Filed: Mar. 23, 2007
(86) PCT No.: PCT/GB07/01058

§ 371 Date: Jun. 29, 2009

(30) Foreign Application Priority Data
Mar. 24, 2006 (GB) 0605961.2

(51) Int. Cl. A61B 17/70

(52) U.S. Cl. 606/249

(57) ABSTRACT

A device for insertion into a space between adjacent processes of vertebrae comprising a flexible enclosure which can be placed in a collapsed form prior to insertion between adjacent processes of vertebrae and subsequently placed in an expanded form by injection of a settable resin under pressure, wherein surfaces of the device facing the adjacent processes of vertebrae are closer together in said collapsed form and further apart in said expanded form.

This invention relates to the insertion of one or more spacing means in the human vertebral column and is an improved means of provision and insertion of such spacing means.

Recent advances in minimally invasive spinal surgery have led to the adoption of spacing means in order in increase the distance between adjacent spinous processes extending from the rear of the spinal vertebrae. Such spacing means are presently marketed by U.S. companies such as Medtronics Inc. and Saint Francis Medical Inc.

An example of a known spacing means is disclosed in WO2005/009300A. This discloses spacing means which can be inserted between adjacent spinous processes. The spacing means has a central spacer section which is inserted between adjacent spinous processes and expandable end sections located on either side of the central section. The spacer is inserted between adjacent spinous processes so that the centre station lays between the spinous processes and the end sections are expanded so that the end sections extend on either side of the adjacent spinous processes and retain the spacing means in place.

These known spacing means suffer a distinct disadvantage in that due to their size and other considerations they are not suitable for use in the cervical spine. Further the said spacing means each require the creation of a significant surgical trauma in order to provide access for the insertion of the said spacing means into the human body.

It is the object of the present invention to provide an improved means of separating the adjacent vertebral processes which has a minimal cross-sectional profile during insertion so that both the time taken for the surgical procedure, and the trauma caused by the procedure, are reduced.

The present invention provides a device for insertion into a space between adjacent processes of vertebrae comprising a flexible enclosure which can be placed in a collapsed form prior to insertion between adjacent processes of vertebrae and subsequently placed in an expanded form by injection of a settable resin under pressure, wherein surfaces of the device facing the adjacent processes of vertebrae are closer together in said collapsed form and further apart in said expanded form.

The present invention consists of a device including a flexible enclosure. The device of the invention can be inserted into the space between adjacent vertebral processes in a collapsed condition using minimally invasive surgery. Following the correct positioning of said device, said flexible enclosure is expanded into a desired form by means of injection of a pre-polymer material delivered under appropriate pressure along a catheter which is connected to said device and by means of which said device is positioned between said adjacent spinous processes.

Accordingly, the invention provides a solution to the problem of surgical trauma caused by inserting the device because the device is inserted in a collapsed form smaller than the required separation of the adjacent processes of vertebrae and expanded to a size defining the required separation after insertion.

Further, the device can be expanded to a required size by control of the applied pressure. As a result, the device can be more precisely arranged to provide a required separation between adjacent processes and a smaller range of sizes of devices is required to be stocked.

Further embodiments of the invention will now be described by way of example only with reference to the accompanying diagrammatic figures in which:

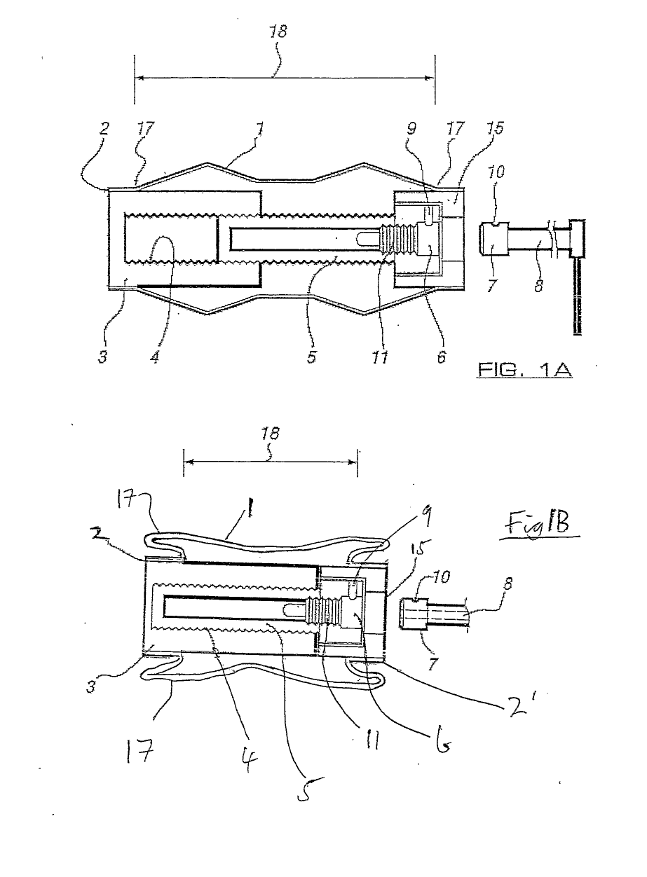

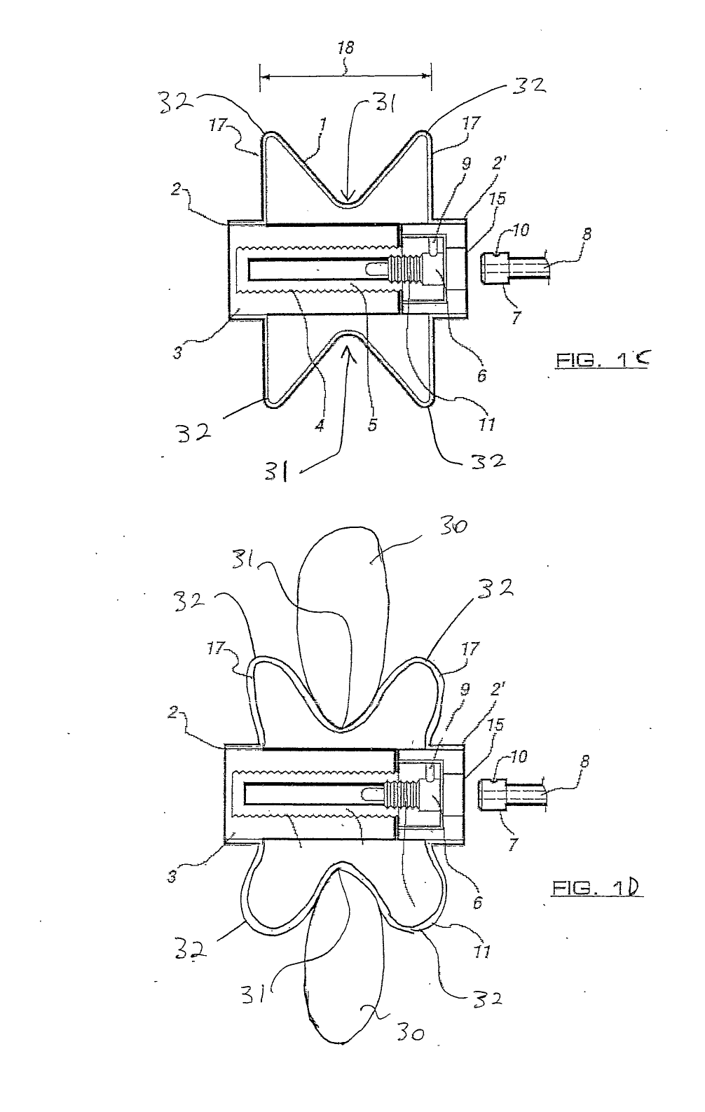

FIG. 1a shows a spacer device according to the invention in a collapsed condition.

FIG. 1b shows the device of 1a in intermediate condition.

FIG. 1c shows the device 1a in an expanded condition in isolation.

FIG. 1d shows the device of 1a in an expanded condition in practise.

FIG. 1e shows detail of a valve used in the device for FIG. 1a, and

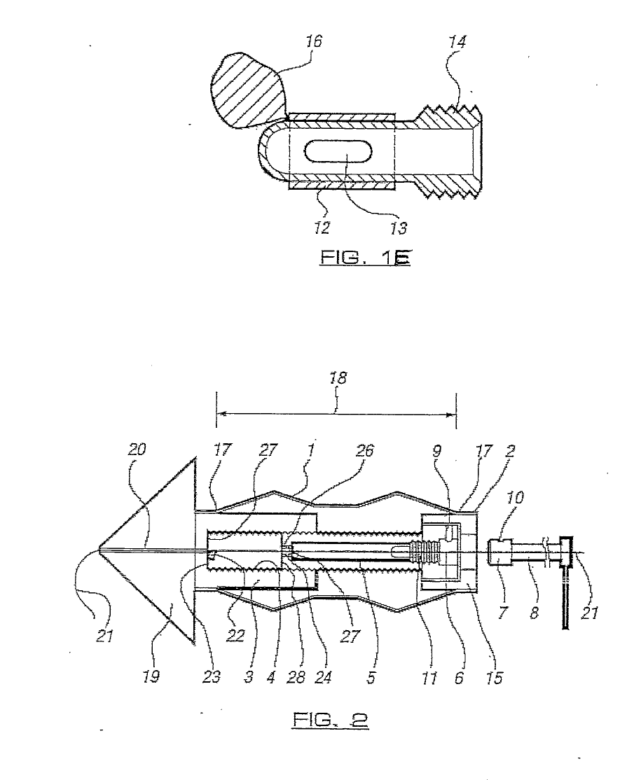

FIG. 2 shows an alternative embodiment of the device.

A first embodiment of the device is now described by reference to FIGS. 1a to 1e. FIG. 1a shows the device of the invention in a compact condition prior to insertion between the adjacent processes of the spinous vertebrae. At this point, said flexible enclosure 1 is stretched along a central tubular component so that it's cross-sectional profile is reduced. FIG. 1b shows the device of the invention is an intermediate form after the stretching has been released. FIG. 1c show the device expanded in isolation. FIG. 1d shows the device of the invention with the said flexible enclosure 1 expanded to its final form following positioning between adjacent vertebrae.

Referring to FIG. 1a, one end of the said flexible enclosure 1 is bonded, clamped or otherwise attached at 2 to the end of a first central tubular component 3. The said first central tubular component 3 is provided with an internal threaded portion 4 into which a second externally threaded tubular component 5 is screwed. The said second tubular component 5 is provided at one end with an aperture 6 which is shaped so as to receive the end 7 of a catheter 8 which may be entered into the said shaped aperture 6. Said end 7 of said catheter 8 is secured in said shaped aperture 6 by means of a sprung ball 9 provided in the wall of the aperture 6, which engages with a depression 10 in said end of said catheter 8. Said end of said catheter 8 may be removed from the said shaped aperture 6 by sudden application of a withdrawal force in a direction parallel to the axis of said catheter 8. If desired, an alternative means of removably engaging said catheter 8 in said aperture 6 may be adopted such as for example a bayonet fitting. A threaded removable one-way valve 11 (shown in greater detail in FIG. 1e) is provided within the bore of said second tubular component 5. Any suitable valve mechanism may be used, the example in FIG. 1e being comprised of a tight rubber sleeve 12 covering an aperture 13 in a further tubular component 14 through which a viscous mixture of catalysed prepolymer 16 such as poly vinyl siloxame may be passed under pressure to enter the interior space of said tubular components 3 and 5 and pass therefrom into the void contained by the flexible enclosure 1.

The non-threaded end of the second tubular component 5 is rotatably seated in a housing 15 through which an axial clearance hole is provided for passage of the shaped end 7 of the catheter 8 or a driving tool (8a in FIG. 1a) into the said shaped aperture 6 provided in said tubular component 5. The second end of said flexible enclosure 1 is bonded or otherwise attached to the housing 15 at 2.

Before insertion into the body, the device is set in the compact condition shown in FIG. 1a by inserting the shaped end 7 of a tool 8 into the shaped cavity 6 provided in the second tubular component 5 and rotating said second tubular component 5 in an anticlockwise direction so as to screw said second tubular component 5 outwardly from said first tubular component 3 thereby extending the length 18 of said central tubular section between the ends 17 of said flexible enclosure 1.

The surgical procedure for the insertion of the device may now be carried out by passage of said device in its compact condition, with catheter attached, along a cannula (not shown) which has ben entered into the body of the patient so as to facilitate the positioning of device of the invention contained within said cannula between the adjacent spinous process which it is desired to separate. Said cannula may now be partially withdrawn from the body while said catheter is maintained in a stationary position so as to release said device from said cannula. Upon positioning of said device in compact condition between said adjacent spinous processes the length of the central tubular assembly of the spacing means is reduced by rotation of said catheter 8 end in a clockwise direction so as to screw said second tubular component 5 inwardly into the first tubular component 3. Following said reduction in length of said tubular assembly, said flexible enclosure 1 will be prevented from assuming its natural form by the presence of the adjacent spinous processes and surrounding tissue, as shown in FIG. 1b.

As shown in FIG. 1b, although the length 18 of the tubular assembly is reduced by the clockwise rotation of the catheter 8 end of the assembly there is no corresponding increase in the thickness or length of the assembly. At this time the flexible enclosure 1 is not pressurised and so the flexible enclosure 1 is simply held between the tubular assembly and the adjacent spinous processes and surrounding tissue.

The flexible enclosure 1 may now be placed in a desired form by injection under pressure of a viscous mixture of a pre-polymer and catalyst required for further polymerization of said pre-polymer. Said viscous mixture is entered into the bore of said catheter 8 and driven down the bore of said catheter 8 for delivery through said one way valve 11 from a trigger operated, mixing and pressurizing dispenser (not shown), familiar to those skilled in the art of dentistry.

Upon completion of the pressurizing procedure the catheter 8 may be withdrawn from the shaped cavity 6 of the spacing means by application of a sharp withdrawal motion in an outward direction along the axis of said catheter 8. Said catheter 8 may then be removed from the body.

As shown in FIG. 1c, if pressurised in isolation, outside of the body, the final form of the flexible enclosure 1 forms a substantially U or V shaped profile on each side of the spacing means with a central part 31 extending a relatively shorter distance from the tubular components located between two end sections 32 extending relatively further from the tubular components. In use, the device is located between adjacent spinous processes so that the adjacent spinous processes lay within the central part 31 of the U or V shaped profile.

An example of the spacer device in place between two spinous processes 30 is shown in FIG. 1d.

As shown in FIG. 1d, because the flexible enclosure 1 is flexible the profile of the central section 31 of the flexible enclosure 1 will conform to the profile of the opposing surfaces of spinous processes 30 while the end parts 32 of the flexible enclosure 1 tend to extend outwardly to the sides of the spinous processes 30.

It will be understood that although the spacer device is embedded within the tissues of the patients body, these tissues offer far less residence to, and so have less influence on the shape of, the flexible enclosure 1 than the bone of the spinous processes 30.

When the polymerization has been completed the central section 31 of the spacing means located between the spinous processes 30 will control the separation of the spinous processes 30 while the end parts 32 of the flexible enclosure 1 extending to the sides of the spinous processes 30 will retain the spacer in place.

The degree of expansion outwardly of the central section of flexible enclosure 1 and the force exerted on the adjacent spinous processes 30 by the flexible enclosure 1 during the pressurising procedure will depend upon the pressure within the flexible enclosure 1.

In a preferred procedure, after the device as been placed between adjacent spinous processes 30 the catheter 8 is used to pressurise the enclosure 1 with a fluid at a pressure which is varied while the expansion of the flexible enclosure 1 and separation of the adjacent spinous processes 30 is measured. The pressure corresponding to the desired degree of expansion of the flexible enclosure 1 and separation of the adjacent spinous processes 30 is measured, and the fluid is then withdrawn.

The flexible enclosure 1 is then repressurised to the said measured pressure corresponding to the desired expansion and/or separation with the mixture of pre-polymer and catalyst as discussed above so that when the polymerizing process has been completed the size of the spacer and separation of the adjacent spinous processes 30 is exactly as required.

In principle this two stage process of pressurisation and measurement with a non-polymerising fluid and a polymerising mixture could be replaced by a single stage process in which the flexible enclosure 1 is pressurised using the polymerizing mixture and the expansion of the flexible enclosure 1 and separation of the adjacent spinous processes 30 measured so that the pressure of the mixture and degree of expansion of the flexible enclosure 1 can be adjusted to the desired value before the polymerisation process is completed. However, such a one stage process can give rise to problems that the pressurisation and measurement stage of the procedure is placed under an undesirable time limit by the requirement to complete the procedure before the polymerisation of the mixture makes it solid.

In one particularly preferred approach the first stage of pressurisation and measurement is carried out using the uncatalysed pre-polymer and the subsequent pressurisation is carried out using a mixture of said pre-polymer and a catalyst.

In order to allow the expansion of the flexible enclosure 1 to be controlled and predictable it is preferred for the flexible enclosure 1 to be flexible but not elastic. Accordingly, the flexible enclosure 1 can be formed of a material which is flexible but substantially non-elastic so that the area of the surface of the flexible enclosure 1 does not significantly vary with changes in the pressure and only the shape of the enclosure 1 varies.

However, the flexible enclosure 1 may alternatively be formed of an elastic material.

The viscous catalysed pre-polymer used to expand the spacing means under pressure may comprise any suitable polymerisable mixture with appropriate flow characteristics which will after curing within an acceptable period of time provide a solid elastic core within said spacing means thereby restoring and maintaining the desired form of such spacing means positioned between said spinous processes of adjacent vertebrae.

The above description of use of the spacer device according to the invention includes a process in which the device is first pressurised while the expansion of the device is measured and subsequently depressurised and repressurised to the same pressure with polymerizing mixture. As an alternative to use of a device which is pressurised, depressurised and repressurised with different fluids it may be preferred to insert a first device and pressurise it while measuring the degree of expansion, then depressurise and remove the first device, and finally insert a new device and pressurise it to the desired pressure with a polymer mixture. This procedure is more complex, but may be preferred in order to avoid any possible problems of contamination of the polymerizing mixture by the fluid used for the earlier pressurisation with measurement stage.

A second embodiment of the invention shown in FIG. 2 may be inserted into the space between adjacent spinous processes prior to expansion using a guide wire positioned to pass through the said space before insertion of the device of the invention. FIG. 2 shows the device in compact form prior to insertion. In this embodiment the first tubular component 3 extends outwardly and away from the said expandable enclosure 1 by means of a conical extension 19 which is provided with an axial hole 20 of sufficient diameter to permit the passage of a guide wire 21 through said hole. A displaceable flap 22 is situated over the hole 20 and fixed at one point 23 to the internal face 27 of the left hand end of the first tubular component 3 to act as a closure of the hole 20 following removal of the guide wire. A similar hole 24 is provided to permit passage of said guide wire through the end of second tubular component 5 and a similar flap 25 is provided within said second tubular component 5 to provide a seal over said guidewire hole in said second tubular component 5. Alternatively said guidewire hole in said first or second tubular component 3 or 5 may be offset (shown dotted at 26) from the axis of said tubular component so that upon advance of second tubular component 5 along said first tubular component 3 following removal of said guidewire, contact between internal face 27 of said first tubular component 3 and external face 28 of said second tubular components provides an effective seal over both holes.

The surgical procedure for the insertion of the second embodiment of the device may be carried out as follows. A guide 21 is inserted into the body of the patient and fed through the space between the adjacent spinous processes between which it is desired to insert the device of the invention using guidance systems familiar to those experienced in the surgical art. The device of the invention in compact condition with the catheter 8 removably attached is fed along said guidewire until it is positioned appropriately between said adjacent spinous processes. Said guide wire is then removed through said catheter 8. Said flexible enclosure may now be placed in the desired form by first reducing the length of the said central tubular section followed by injection of catalysed prepolymer as described in respect of said first embodiment of the invention. Following fixing of the form of the flexible enclosure, the catheter 8 may be removed from the body as previously described and the wound closed in the normal manner.

In the embodiments described above the flexible enclosure is collapsed by axial extension driven by mutual rotation of threaded cylindrical components of the device. Alternative mechanisms to axially extend the device to collapse the flexible enclosure could be used. The skilled person will have no difficulty in devising suitable mechanisms.

In all of the embodiments of the invention, in order to ensure the fullest possible collapse of the flexible enclosure to minimise the width of the device in the collapsed form, and so minimise the surgical trauma required for insertion of the device, the interior of the flexible enclosure may be subject to a vacuum. In the described embodiments this vacuum can be conveniently applied using the catheter.

Optionally the mechanism used to place the device in the collapsed condition may also be vacuum operated so that the application of the vacuum to the device axially extends the device to place the device in the collapsed condition and also collapses the flexible enclosure inwardly as far as possible. In this arrangement the mechanism is arranged to leave the collapsed condition on release of the vacuum. Alternatively, the mechanism can be arranged to move the device out of the collapsed condition in response to pressurising of the flexible enclosure with a fluid.

In one preferred embodiment the first and second tubular sub components used in the embodiments described above are not threaded and are arranged so that changes in the length of the device can be carried out by relative sliding movement of the tubular components. The device can then be changed in length and be driven between the collapsed and expanded conditions by selectively applying different pressures to the interior of the flexible enclosure. For example, a vacuum can be applied to move the device into the collapsed position. The device can subsequently be driven into the expanded position by injecting a pressurised fluid into the flexible enclosure. This pressurised fluid could be a non polymerising fluid or a polymerising mixture depending which of the methods of installation described above is used. This arrangement is particularly effective if the flexible enclosure is formed of a material which is flexible, but substantially not elastic.

The flexible enclosure provided in both embodiments of the invention may vary in wall thickness in different areas in order to facilitate establishment and maintenance of the desired form of the enclosure. Said flexible material of said enclosure may be provided with reinforcement in the form of plastic or other filaments which may be disposed so as to provide differential strength in different areas of said enclosure. by this means the establishment and maintenance of the desired expanded form of said enclosure may be achieved.

In order to facilitate viewing of the device during insertion in the human body, the material of the said flexible enclosure may be rendered radio-opaque by inclusion therein of a radio opaque in forms of familiar to those skilled in the art of radiographic imaging.

The materials of the tubular components of the device may be constructed from stainless steel or other alloys or materials suitable for implantation in the human body. The device may be surface treated in order to minimize any inflammatory reaction of surrounding tissue.

In both embodiments of the invention the one way valve 11 may be dispensed with if the catalysed pre-polymer is maintained under pressure by means of the catheter injection system until polymerization is complete.

1. A device for insertion into a space between adjacent processes of vertebrae comprising a flexible enclosure which can be placed in a collapsed form prior to insertion between adjacent processes of vertebrae and subsequently placed in an expanded form by injection of a settable resin under pressure, wherein surfaces of the device facing the adjacent processes of vertebrae are closer together in said collapsed form and further apart in said expanded form.

2. The device according to claim 1, wherein the flexible enclosure is attached to a central tubular assembly.

3. The device of claim 2, wherein the central tubular component is comprised of two or more tubular sub-components.

4. The device of claim 3, wherein one of said tubular sub-components is externally threaded and is capable of movement along the common axis of the components into and out of a threaded bore of the other tubular sub-component.

5. The device of claim 1, wherein said flexible enclosure is collapsed by applying a vacuum.

6. The device of claim 4, wherein the flexible enclosure is collapsed towards its axis by outward axial movement of said externally threaded tubular sub-component along the bore of said other tubular sub-component.

7. The device of claim 1, wherein, when the device is in said expanded form, the flexible enclosure has a shape with a central section located between two end sections, the end sections extending outwardly further than the central section.

8. The device of claim 7, wherein, when the device is between adjacent processes of vertebrae in said expanded form, the central section contacts said processes of vertebrae.

9. The device of claim 1, wherein said suitable resin is a catalysed pre-polymer.

10. The device of claim 1, wherein the components of said central tubular assembly have respective central axial holes to permit movement of the device over a guidewire.

11. The device of claim 1, wherein said device further comprises means for removably attaching a catheter thereto.

12. The device of claim 11, wherein said removable catheter has a shaped end which fits a mating cavity in one of said tubular sub-components to enable axial rotation of said sub-component.

13. The device of claim 12, wherein the said flexible enclosure is collapsed towards said axis of said central tubular assembly by application of a vacuum down the bore of said catheter in addition to the extension of the combined length of said combined central assembly.

14. The device of claim 1, wherein the wall thickness of said flexible enclosure varies in different areas thereof.

15. The device of claim 1, wherein the material forming the said flexible enclosure is reinforced using filaments of plastic or other material.

16. The device of claim 1, wherein the enclosure and other components are manufactured from materials suitable for implantation in a human or animal body.

17. The device of claim 1, wherein the surfaces of the device are treated so as to minimize inflammation or other adverse effects on surrounding tissue.

18. The device of claim 1, wherein the material of said flexible enclosure is rendered radio-opaque by inclusion therein or attachment thereto of a radio opaque material.

19. The device of any of the above claims, wherein the vertebrae are human vertebrae.
